(19) United States
(12) Patent Application Publication
(10) Pub. No.: US 2015/0253308 A1
(43) Pub. Date: Sep. 10, 2015

(54) COMPOSITIONS AND METHODS OF TREATING GLAUCOMA

(71) Applicant: ORASIS, Irvine, CA (US)

(72) Inventor: Richard A. Hill, Irvine, CA (US)

(21) Appl. No.: 14/720,627

(22) Filed: May 22, 2015

Related U.S. Application Data
(62) Division of application No. 13/887,217, filed on May 3, 2013.

(51) Int. Cl. G01N 33/50

(52) U.S. Cl. CPC G01N 33/5082

(57) ABSTRACT

Methods are disclosed for treating glaucoma by treating a novel target. Methods for treating glaucoma by restoring the filtration capabilities of the endothelial lining of Schlemm's canal are provided. A method for identifying compounds capable of restoring the filtration capability of the juxtacanalicular meshwork is also provided.

### INCORPORATION BY REFERENCE TO ANY PRIORITY APPLICATIONS

This application is a divisional of U.S. application Ser. No. 13/887,217, filed May 3, 2013, titled “COMPOSITIONS AND METHODS OF TREATING GLAUCOMA,” the entirety of which is incorporated herein by reference.

### BACKGROUND OF THE INVENTION

1. Field of the Invention

Aspects of the invention relate generally to methods and compositions for treating glaucoma. More particularly, the treatment of glaucoma may involve restoring the filtration capability of the trabecular meshwork of the eye. A method of drug screening is also disclosed.

2. Description of the Related Art

Glaucoma is a leading cause of blindness characterized by increased pressure within the eye, which, if untreated, can lead to destruction of the optic nerve. A clear fluid called aqueous humor is formed constantly by the ciliary bodies and secreted into the posterior chamber. This fluid passes over the lens and enters the anterior chamber. Aqueous humor passes out the anterior chamber of the eye at approximately the same rate at which it is produced through one of two routes. Approximately 10% of the fluid percolates between muscle fibers of the ciliary body, and approximately 90% of the fluid is removed via the “canalicular route,” through a filter-like mass of tissue called the trabecular meshwork and Schlemm's canal, and then enters the scleral venous network.

There are a number of different forms of glaucoma, including open-angle and closed-angle glaucoma, as well as steroid induced glaucoma. The most common form of glaucoma is open-angle, which results from increased resistance in the outflow pathway through the trabecular meshwork. The mechanism by which the outflow pathway becomes blocked or inadequate is poorly understood, but the result is an increase in pressure within the eye, which compresses the axons in the optic nerve and can compromise vascular supply to the nerve. Over time, this can result in partial or total blindness. The trabeculae are not physically obstructed, but no longer efficiently transport fluid between the anterior chamber and the scleral drainage veins.

Current treatment of glaucoma is either medical, surgical, or both. Medications for the treatment of glaucoma include prostaglandin analogs which increase fluid percolation between muscle fibers of the ciliary body and miotics which are administered as drops and cause contraction of the pupil of the eye by tightening the muscle fibers of the iris to increase the rate at which the aqueous humor leaves the eye. Epinephrine drops have also been successful in reducing intraocular pressure, but have significant side effects. Other medications are employed, such as β-adrenergic blocking agents as drops or carbonic anhydrase inhibitors as pills, which reduce the production of fluid.

Surgical solutions include applying a laser to multiple spots along the trabecular meshwork, which is thought to change the extracellular material and enhance outflow. Approximately 80% respond initially to this treatment, but, unfortunately, 50% have increased pressure within five years. Other solutions attempt to increase the permeability of the trabecular meshwork or widen Schlemm's canal. Another surgical procedure is a trabeculectomy, wherein an incision is made in the conjunctiva to form a hole in the sclera for aqueous fluid to flow through. This can be performed either with a laser or through an open procedure. Both routes have risks, including infection or injury to the eye. With either route, frequently that the hole closes up over time with consequent increase in pressure. A variety of apparatuses have been suggested, such as implantation as a shunt or drain across the trabecular network, draining either to the sclera or to Schlemm's canal. Alternatively, some treatments have targeted the pores between endothelial cells lining Schlemm's canal.

However, a need remains for a way of safely, lastingly, and effectively treating open-angle glaucoma. Current medical and surgical treatment options often lose their efficacy with time. Furthermore, surgical treatments have associated risks of infection or injury to the eye, and current medical solutions often come with significant side effects either affecting vision, the structures of the eye, or with systemic side effects. A need also exists for a treatment of glaucoma which addresses the underlying pathology in the aqueous humor outflow system and leads to return of drainage as seen in non-glaucomatous eyes. There exists a need as well for improved models of testing drugs ex vivo for use in this ophthalmic application.

### SUMMARY OF THE INVENTION

Methods and compounds for the treatment of glaucoma are provided. The invention is based on the discovery of ascorbic acid conjugated phospholipids in ocular tissue, which provides a target for decreasing intraocular pressure.

In some embodiments, a method of lowering intraocular pressure is provided. The method comprises administering a therapeutically effective amount of an amphipathic compound or pharmaceutically acceptable derivative thereof to a subject in need of such treatment, wherein the amphipathic compound comprises ascorbic acid. In one embodiment, the amphipathic compound comprises an ascorbic acid head and a nonpolar tail.

For example, a method of lowering intraocular pressure comprising administering a therapeutically effective amount of a compound comprising phosphorylated ascorbic acid or pharmaceutically acceptable derivative thereof to a subject in need of such treatment is provided. In one embodiment, the compound comprises a phospholipid. In another embodiment, the compound comprises micelles, or is configured to form micelles under favorable conditions. In another variation, the compound forms a micelle after administering the compound to the subject.

In another example, a method of lowering intraocular pressure comprising administering a therapeutically effective amount of a glycolipid comprising an ascorbic acid head or pharmaceutically acceptable derivative thereof to a subject in need of such treatment is provided.

In another variation, a method is provided for lowering intraocular pressure comprising administering to a subject in need of treatment a therapeutically effective amount of an enzyme or enzymatically active fragment thereof having an enzymatic activity of forming an amphipathic compound. In one embodiment, the enzymatic activity is a kinase activity. In another embodiment, the enzymatic activity is an esterase activity. In another embodiment, the enzyme or enzymatically active fragment thereof is delivered by administering a nucleic acid that encodes for the enzyme or enzymatically active agent.

In another embodiment, a method of lowering intraocular pressure is provided. The method comprises administering to a subject in need of treatment a therapeutically effective amount of a nucleic acid that encodes a therapeutic agent. In some embodiments, the therapeutic agent is an enzyme having an enzymatic activity of forming an amphipathic compound. In variations, the enzymatic activity may be a kinase or esterase activity.

A method is also provided of maintaining a target intraocular pressure, comprising: measuring a baseline intraocular pressure of a subject, determining the target intraocular pressure for the subject, selecting a first therapeutic compound which generally decreases intraocular pressure by an amount about the difference between the baseline intraocular pressure and the target intraocular pressure, administering the first therapeutic compound, measuring the intraocular pressure of the subject after administration of the compound, selecting a second therapeutic compound which decreases the intraocular pressure by an amount different than that of the first therapeutic compound if the intraocular pressure after administration is not about the target intraocular pressure; and administering the second therapeutic compound. Such steps are carried out by methods known to one skilled in the art. An amount “about the difference” or “about the target intraocular pressure” is used in this context to mean an amount equivalent to, substantially equivalent to, or equal to +/−1%, 2%, 3%, 5%, 10%, 15%, or 20%. In some embodiments, the therapeutic compound is selected from a group consisting of phosphorylated ascorbic acid, phosphorylated ascorbic acid derivative, ascorbic acid containing phospholipid, an ascorbic acid derivative containing phospholipid, an ascorbic acid containing glycolipid, an ascorbic acid derivative containing glycolipid, or any amphipathic molecule or compound that could allow membrane transport of aqueous humor.

Further provided is a drug screening method comprising: preparing a donor eye tissue, introducing fluid into an anterior segment of the donor eye tissue, introducing a candidate therapeutic molecule into the anterior segment of the donor eye tissue, and determining a measurement related to the introduction of the candidate therapeutic molecule into the anterior segment of the donor eye tissue. In some embodiments, determining a measurement related to the introduction of the candidate therapeutic molecule into the anterior segment of the donor eye tissue comprises measuring an amount or rate of uptake of the candidate therapeutic molecule into the donor eye tissue. In other embodiments, the drug screening method further comprises determining a baseline measurement after introducing fluid into the anterior segment of the donor eye tissue, determining a second measurement after introducing the candidate therapeutic molecule into the anterior segment of the donor eye tissue, and determining the change in the baseline measurement. In some embodiments, the baseline and second measurements are selected independently from the rate of outflow from the anterior segment of the eye, and the pressure maintained in the anterior segment.

### BRIEF DESCRIPTION OF THE DRAWINGS

FIG. 1A is a cross-sectional illustration of the anterior portion of the eye.

FIG. 1B is a cross-sectional illustration of the irido-corneal angle of the eye.

FIG. 2 is a graph showing the MS/MS collision dissociation of d18:1-12:0 glucosyl ceramide standard.

FIG. 3 is a graph showing the LC/MS/MS of +NL 180.2 u for d18:1-12:0 glucosyl-ceramide standard.

FIG. 4 is a graph showing the MS/MS collision dissociation of d18:1-12:0 lactosyl ceramide standard.

FIG. 5 is a graph showing the LC/MS/MS of +NL 342.2 u for d18:1-12:0 lactosyl-ceramide standard.

FIG. 6 is a graph showing brain cerebrosides, BCE-44 run as control for +NL 180 u (hexosyl).

FIG. 7 is an illustration of a proposed structure of an ascorbate conjugate.

FIG. 8 is a graph showing the LC/MS/MS injection +NL 176.2 u (ascorbate sugars) for a methanol blank.

FIG. 9 is a graph showing the LC/MS/MS injection of +NL 176.1 u ascorbate sugars for sample OCG-930.

FIG. 10 is a graph showing the LC/MS/MS injection of +NL 176.1 (ascorbate sugars) for sample OCG-931.

FIG. 11 is a graph showing the sample 930 extract LC/MS/MS of 531.1/176.1 u and 403.4/176.1 u.

FIG. 12 is a graph showing the sample 931 extract LC/MS/MS of 531.1/176.1 u and 403.4/176.1 u.

FIG. 13A is a schematic of the trabecular meshwork and Schlemm's canal; FIG. 13B-C are schematics of the membrane of an endothelial cell in the juxtacannalicular lining of Schlemm's canal.

FIG. 14A is an end on view of a representative cell membrane channel formed by a phospholipid; FIG. 14B is a cross sectional view of a representative cell membrane channel formed by a phospholipid.

FIG. 14C is a representation of a micelle. FIG. 14D is a representation of a reverse micelle.

FIG. 15 is a rendering of a representative phospholipid molecule.

### DETAILED DESCRIPTION OF THE DRAWINGS

### Anatomy

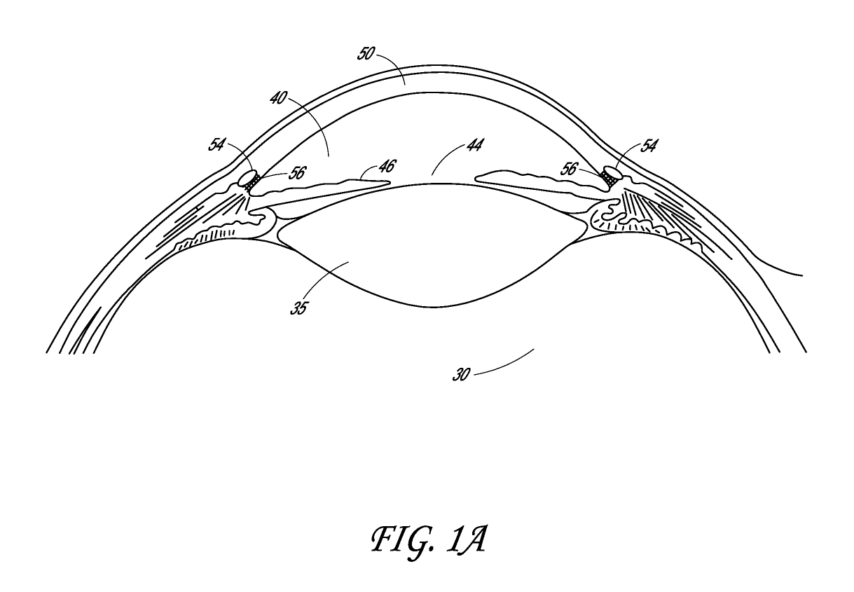

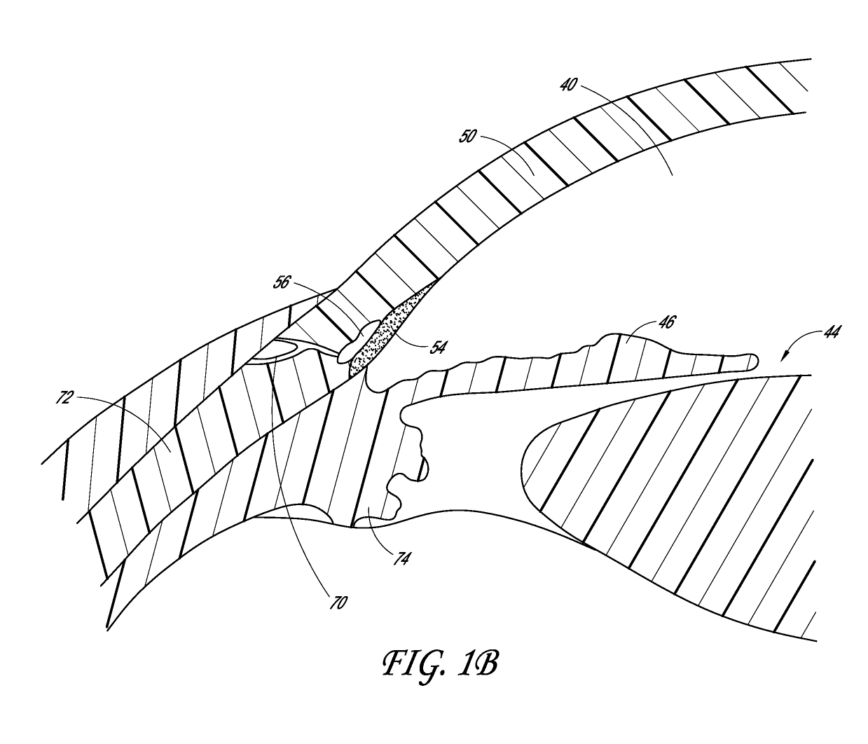

Glaucoma is defined by increased pressure in the chambers of the eye resulting from disordered drainage of the aqueous humor from the anterior chamber 40 (FIG. 1A) of the eye into the aqueous veins 70 (FIG. 1A)_and thence to the scleral venous drainage system. The precise mechanism of drainage is poorly understood. However, it is known that the process, in a normal eye, is energy independent and self-regulating, such that the pressure of the eye remains relatively constant. The outflow rate from the anterior chamber of the eye generally matches the production rate of aqueous humor in the posterior chamber of the eye 30.

The normal eye (FIGS. 1A and 1B), aqueous humor flows through the trabecular meshwork 54 into Schlemm's canal 56, and thereby into the venous system 60 of the sclera 72. The trabecular meshwork 54 and Schlemm's canal 56 are located at the junction between the iris 46 and the sclera 72. The cornea 50, lens 35, and pupil 44 are also visualized. The trabecular meshwork is wedge shaped in structure and runs around the entire circumference of the eye, forming a three dimensional sieve structure. The trabecular meshwork is formed of collagen beams aligned with a monolayer of cells called the trabecular cells, which produce an extracellular substance which fills the spaces between collagen beams. After passing through the trabecular meshwork, aqueous matter crosses the endothelial cells of the Canal of Schlemm 56. In this manner, trabecular meshwork cells and Canal of Schlemm endothelial cells are thought to comprise the cells of the primary outflow pathway of the eye. The trabecular meshwork is suspended between the corneal endothelium and the ciliary body face and is comprised of a series of parallel layers of thin, flat, branching and interlocking bands termed trabeculae. The inner portion of the trabecular meshwork (closest to the iris root and ciliary body 74) is called the uveal meshwork, whereas the outer portion (closest to the Canal of Schlemm) is called the corneoscleral or juxtacanalicular meshwork. The uveal meshwork trabeculae measure approximately 4 μm in diameter, consist of a single layer of cells surrounding a collagen core, and are arranged in layers which are interconnected. The spaces between these trabeculae are irregular and range from about 25 μm to about 75 μm in size. The trabeculae of the corneoscleral meshwork resemble broad, flat endothelial sheets about 3 μm thick and up to about 20 μm long. The spaces between these trabeculae are smaller than in the uveal meshwork and more convoluted. As the lamellae approach the Canal of Schlemm, the spaces between the trabeculae decrease to about 2 μm. The resistance to aqueous humor outflow through the trabecular meshwork has been reported to reside primarily in the juxtacanalicular meshwork (JCM). At this site two cell types are found: trabecular meshwork cells and also endothelial cells of the inner wall of Schlemm's canal. Treatments, both medical and surgical, have attempted to reduce intraocular pressure by increasing the permeability of the trabecular meshwork, creating new outflow pathways, or widening Schlemm's canal. However, these do not adequately address the juxtacanalicular meshwork as the primary source of resistance to outflow.

In contrast to the current level of knowledge regarding cellular processes responsible for aqueous humor production by the ciliary body 74, relatively little is known about the cellular mechanisms in the trabecular meshwork 54 that determine the rate of aqueous outflow. Pinocytotic vesicles have been observed in the juxtacanalicular meshwork and the inner wall of Schlemm's Canal. The function of these vesicles remains unknown, but some investigators have suggested that the bulk flow of aqueous humor through the meshwork cannot be accounted for by flow through the intercellular spaces and that these vesicles play a central role in outflow regulation. Management of outflow by regulation of ion channels in the cell membranes of the juxtacanalicular meshwork and lining of Schlemm's Canal has been proposed. However, it is proposed that a different mechanism, an osmotic drive, is responsible for the regulation of outflow of aqueous humor through the JCM. This osmotic drive is self-regulating, such that changes in intraocular pressure lead to corresponding changes in the rate of outflow so that a relatively constant pressure is maintained.

### Experiment

It is known that the levels of L-ascorbic acid in the aqueous humour (1.06 mmol/l; Arshinoff S. A., et al. “Ophthalmology”, chapter 4.20.2, published by Mosby International Ltd., 1999, herein incorporated by reference in its entirety) are about 20 times higher (Brubaker R. F. et al. “Investigative Ophthalmology & Visual Science”, June 2000, vol. 41, No. 7, pp. 1681, herein incorporated by reference in its entirety) than those present in the blood circulation (20-70 μmol/l, Geigy Scientific Tables, vol. 3, page 132, 8th edition 1985, published by Ciba Geigy, herein incorporated by reference in its entirety). In the case of the retina, the levels of L-ascorbic acid in the eye are actually 100 times higher than those present in the blood circulation.

Studies investigating the levels of ascorbic acid in the glaucomic eye (Pei-fei Lee, M D et al., “Aqueous Humor Ascorbate Concentration and Open-Angle Glaucoma,” Arch Ophthalmol. 1977; 95(2):308-310, herein incorporated by reference in its entirety) and assessing the use of dietary antioxidants in preventing glaucoma (Jae H. Kang et al., “Antioxidant Intake and Primary Open-Angle Glaucoma: A Prospective Study,” Am. J. Epidemiol. (2003) 158 (4): 337-346, herein incorporated by reference in its entirety) show that the level of ascorbic acid did not appear to be predictably reduced in the glaucomic eye, nor does antioxidant use prevent glaucoma. Treatments directed at use of ascorbic acid supplements have been proposed, theorizing that the antioxidant properties may play a role in maintaining reduced intraocular pressure (US2006/0004089, herein incorporated by reference in its entirety).

However, there has not been a satisfactory explanation for the increased levels of L-ascorbic acid in the eye nor an explanation of the role that it plays in maintaining normal function of the eye.

Glycosylated lipids are known to exist in nature. These are typically structural conjugates of monosaccharides, disaccharides and polysaccharides to the glycerol or glycerophospho headgroup of sphingolipids and phospholipids respectively. Phospholipids have hydrophobic fatty acid chains which are linked via a phosphate group to a sugar group. Ogata et al. (U.S. Pat. No. 5,098,898, herein incorporated by reference in its entirety) described synthesis of various phospholipid-type ascorbic acid derivatives by binding a glycerol ester or ether to ascorbic acid via a phosphoric acid residue. Therefore, ascorbate can be a component of phospholipid molecules, and the high levels of ascorbic acid in eye tissue may be explained by its presence as a building block for phosopholipid molecules.

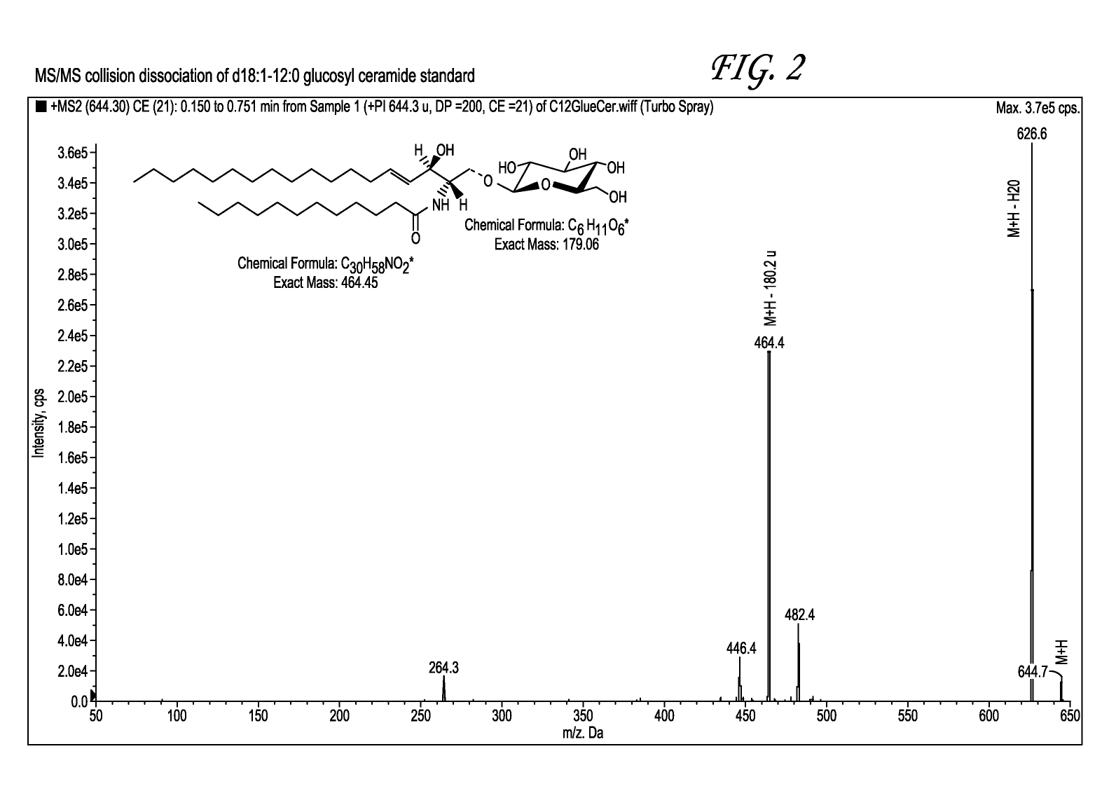

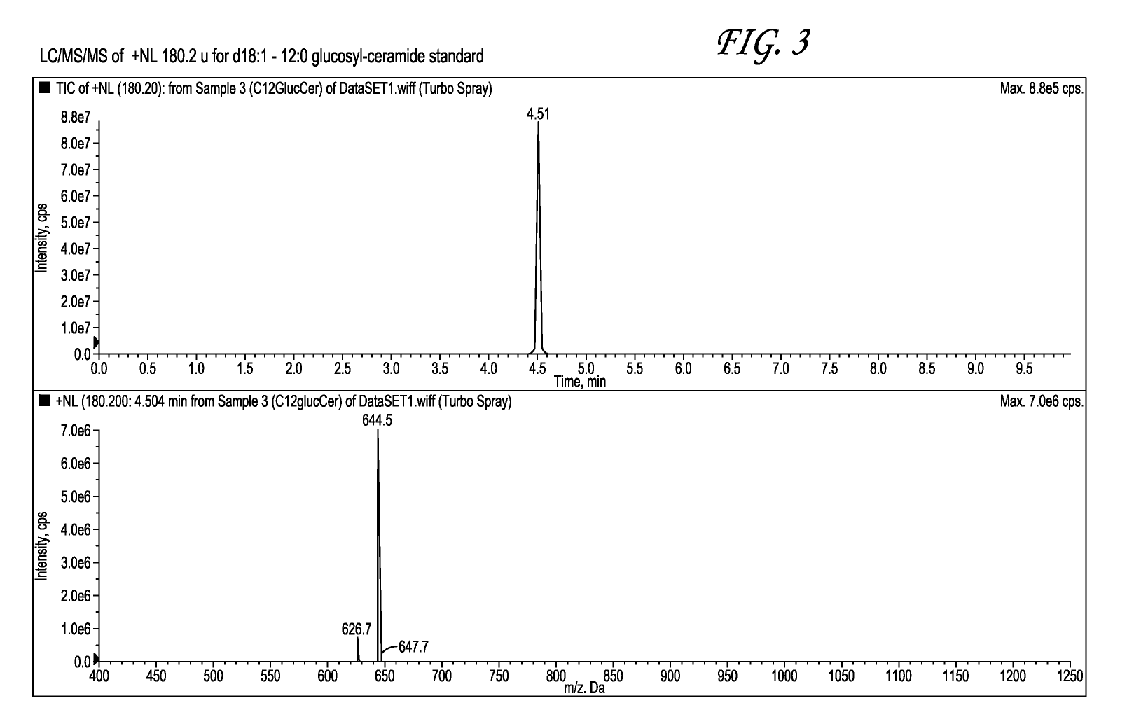

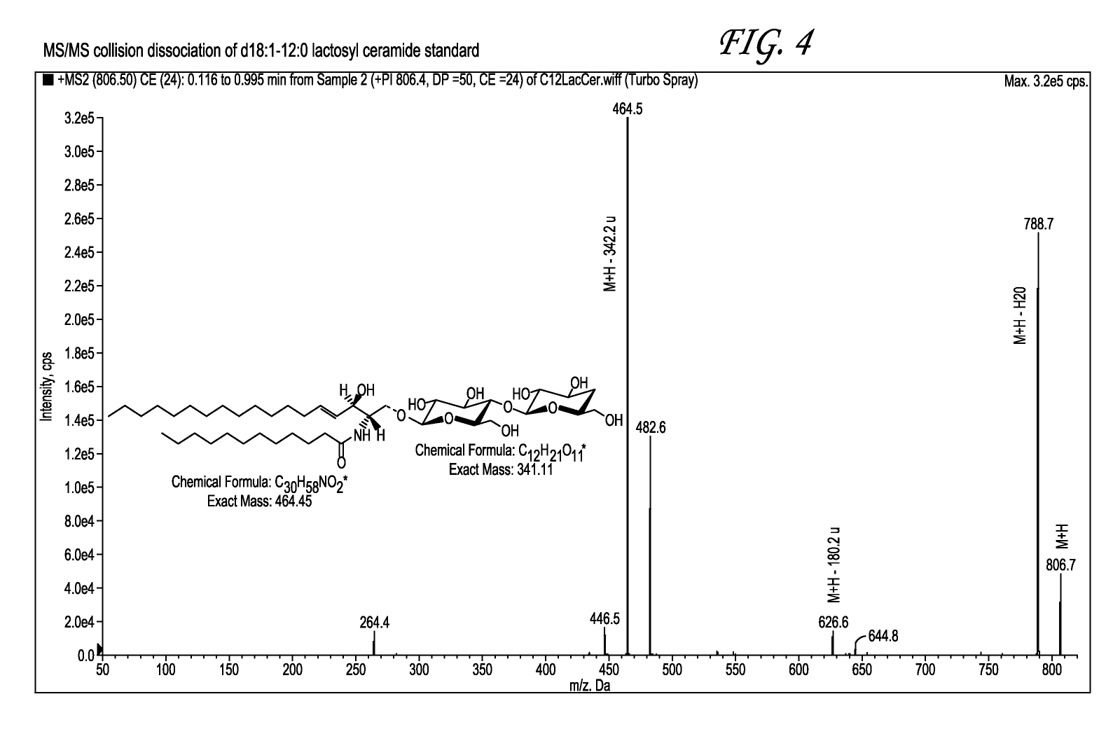

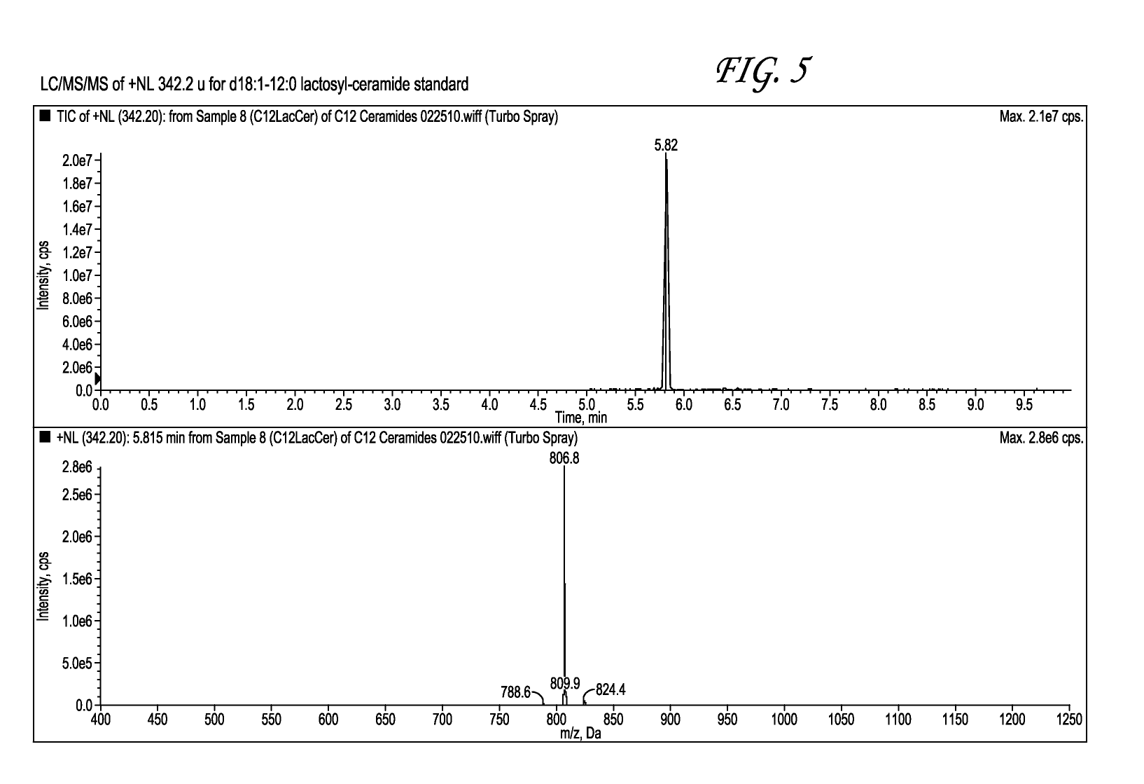

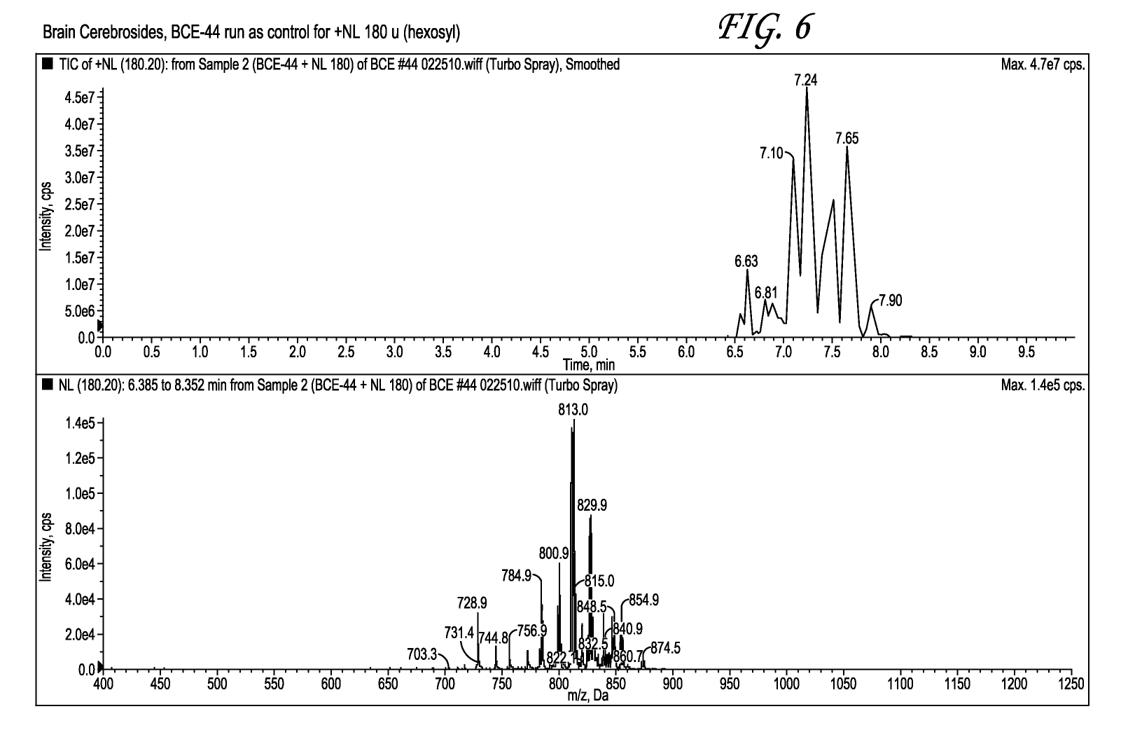

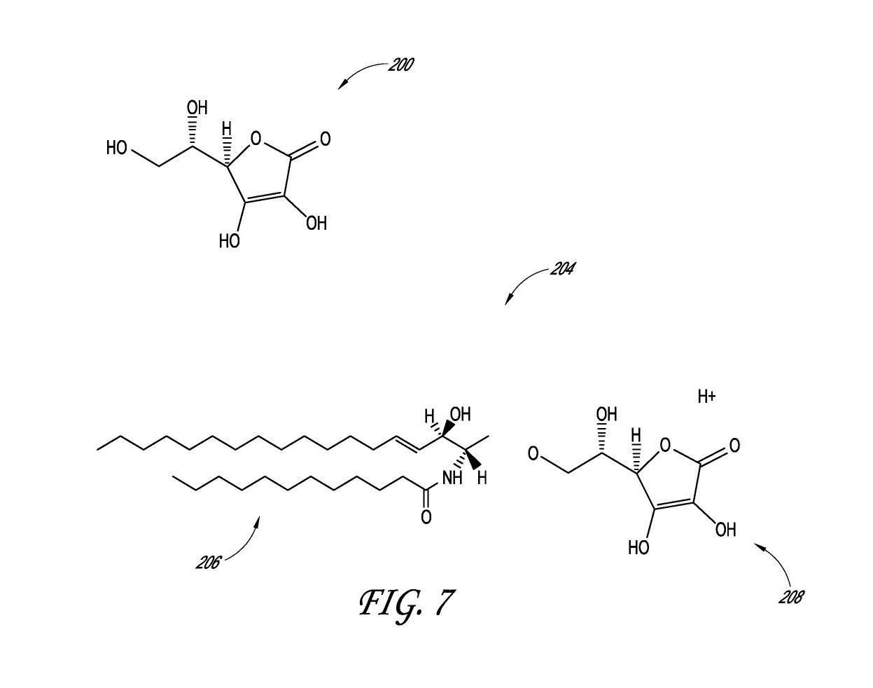

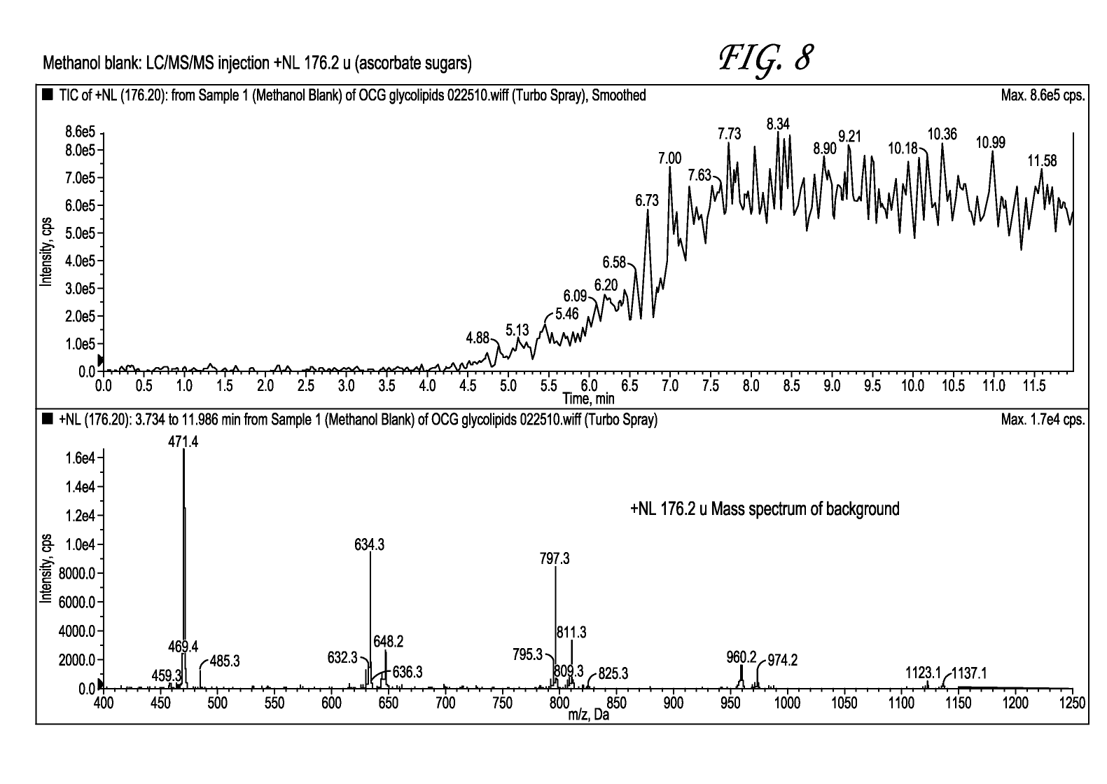

An experiment was designed to confirm the presence of ascorbate conjugated phospholipid compounds in isolated trabecular meshwork tissue samples from non-glaucomatous donors. Tandem mass spectrometry (MS/MS) and liquid chromatography-tandem mass spectrometry (LC/MS/MS) techniques were developed using hexosyl and di-hexosyl standards as surrogates for the proposed detection of ascorbate conjugate structures. Provided eye tissues were then prepared using a common lipid extraction method and solvents for MS analysis. Samples were screened for hexosyl, di-hexosyl and ascorbate structures through precursor ion scanning techniques. To provide screening procedures for the possible discovery of these compounds in eye tissue slices, standard compounds were studied to optimize their detection through neutral loss MS/MS detection from a reversed phase HPLC separation. These neutral loss detection experiments were centered around the loss of 180.2 u for monohexose sugars (FIGS. 2-4, 6), 342.2 u for dihexose (FIGS. 4 and 5) and 176.1 u for ascorbate sugars (FIG. 8). The figure of 176.1 was determined by looking at a proposed structure of an ascorbate conjugate as shown in FIG. 7. Ascorbic acid 200 (FIG. 7) has the chemical formula C₆H₈O₆ with an exact mass of 176.03 and a molecular weight of 176.12. The ascorbate conjugate fragmentation 204 was proposed to occur, leaving a lipid group 206 with formula C₃₀H₅₈NO₂^{•} and exact mass of 464.45 and the ascorbate molecule 208 with formula C₆H₈O₆^{•+} with exact mass 176.03 and molecular weight 176.12.

Trabecular meshwork tissue samples 930 and 931 were received on Jan. 5, 2010 and stored at 2-8° C. until initial extraction was performed on Feb. 25, 2010. The samples were extracted. The entire volume of buffer and tissue was transferred to a 13×100 mm glass test tube. The eye tissue was ground with the end of a glass stirring rod. Tissue was noted to be very fibrous and resistant to disruption. 1.0 mL of HPLC grade methanol was added to the tissue and mixed. The mixture was then sonicated in 37° C. water bath for 1 hour. Next, 1.0 ml of HPLC grade chloroform was added and vortex mixed for 30 strokes on high setting. The mixture was then centrifuged at 2500 rpm for 5 minutes. Next, the bottom layer was transferred to a clean 13×100 mm glass test tube. The top layer was re-extracted with an additional 1.0 mL of HPLC grade chloroform. The mixture was centrifuged as before and resultant lower layer combined with initial lower layer. The chloroform was evaporated to dryness and the resultant material reconstituted with 100 μL of HPLC mobile phase B.

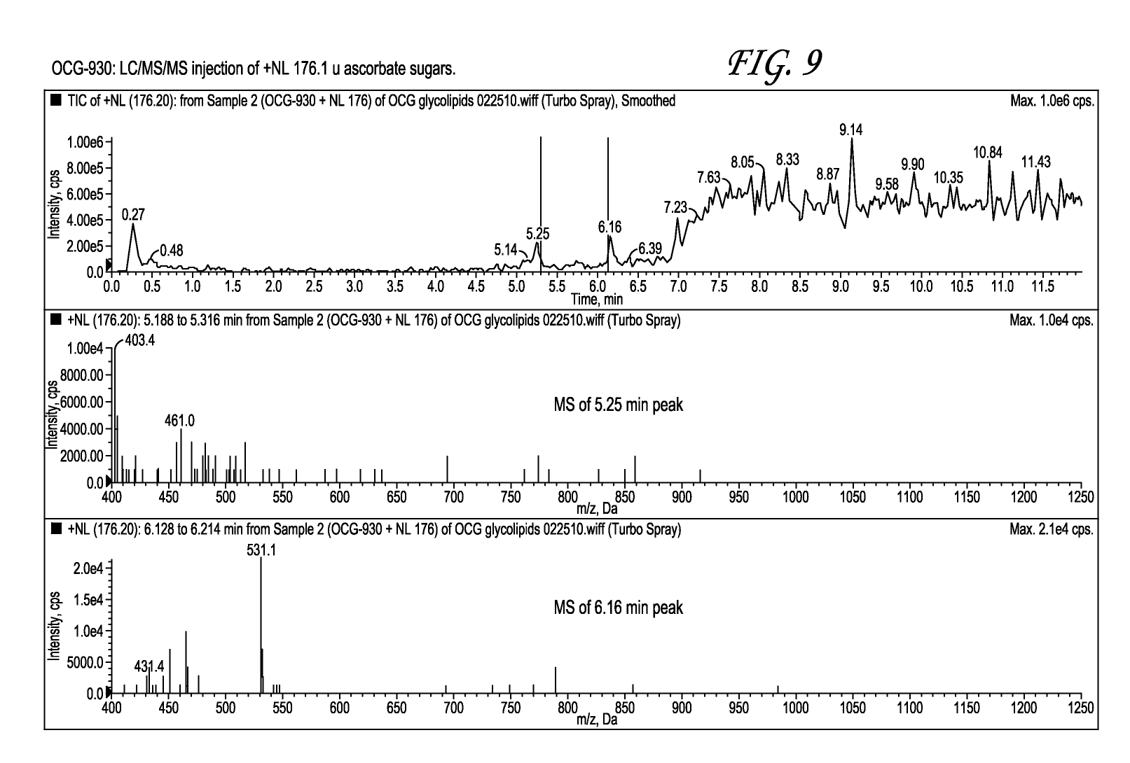

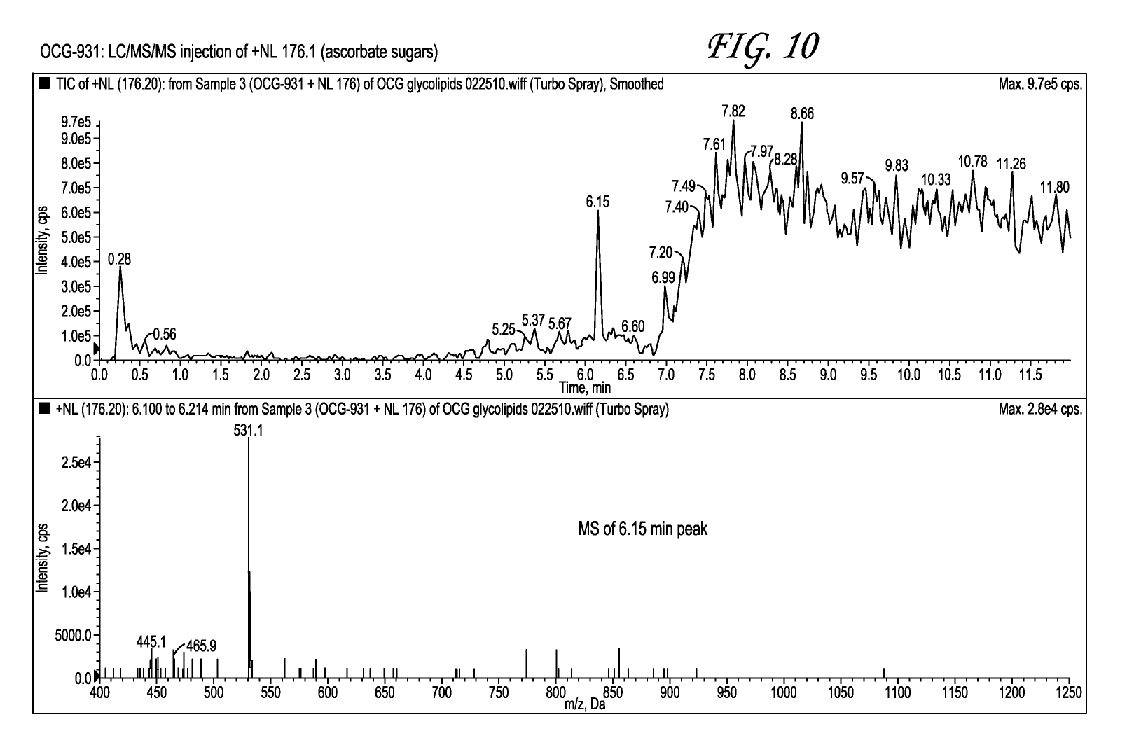

Samples 930 (FIG. 9) and 931 (FIG. 10) were then analyzed via LC/+NL 176.1 u scan. Both samples contained peaks at 5.25 and 6.15 minutes consistent with a compound which exhibits a neutral loss of an ascorbic acid. The parent molecular ions for these peaks were 403.4 u at 5.25 minutes and 531.1 u at 6.18 minutes.

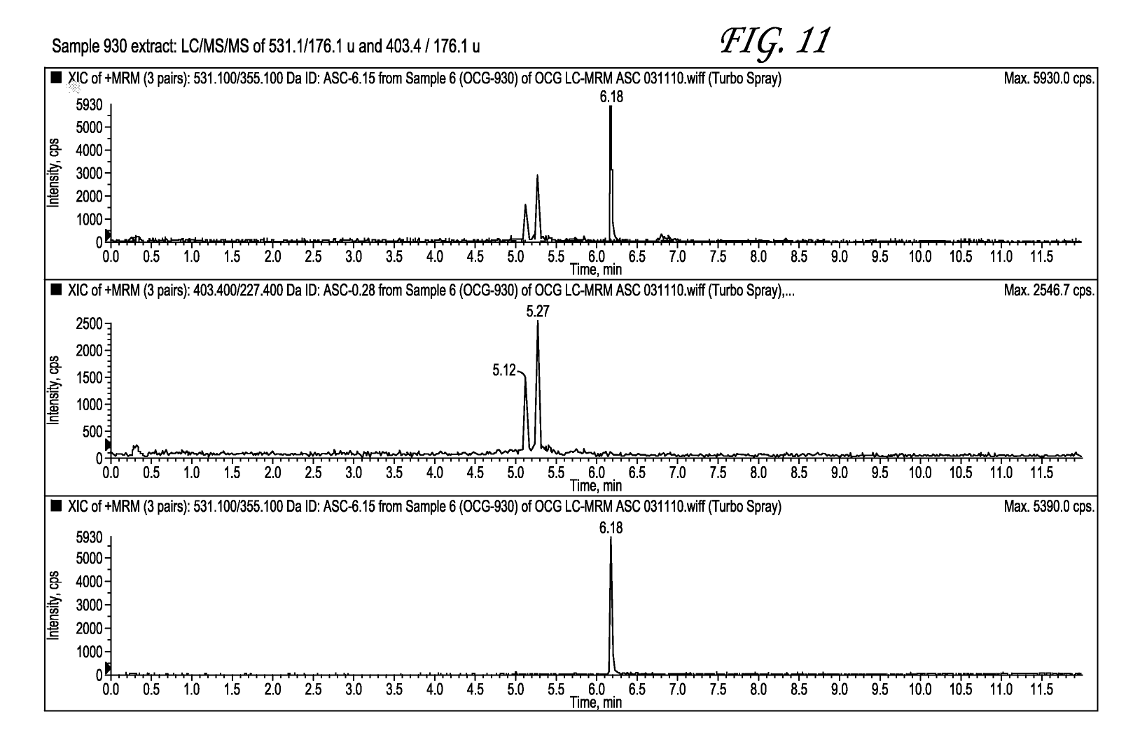

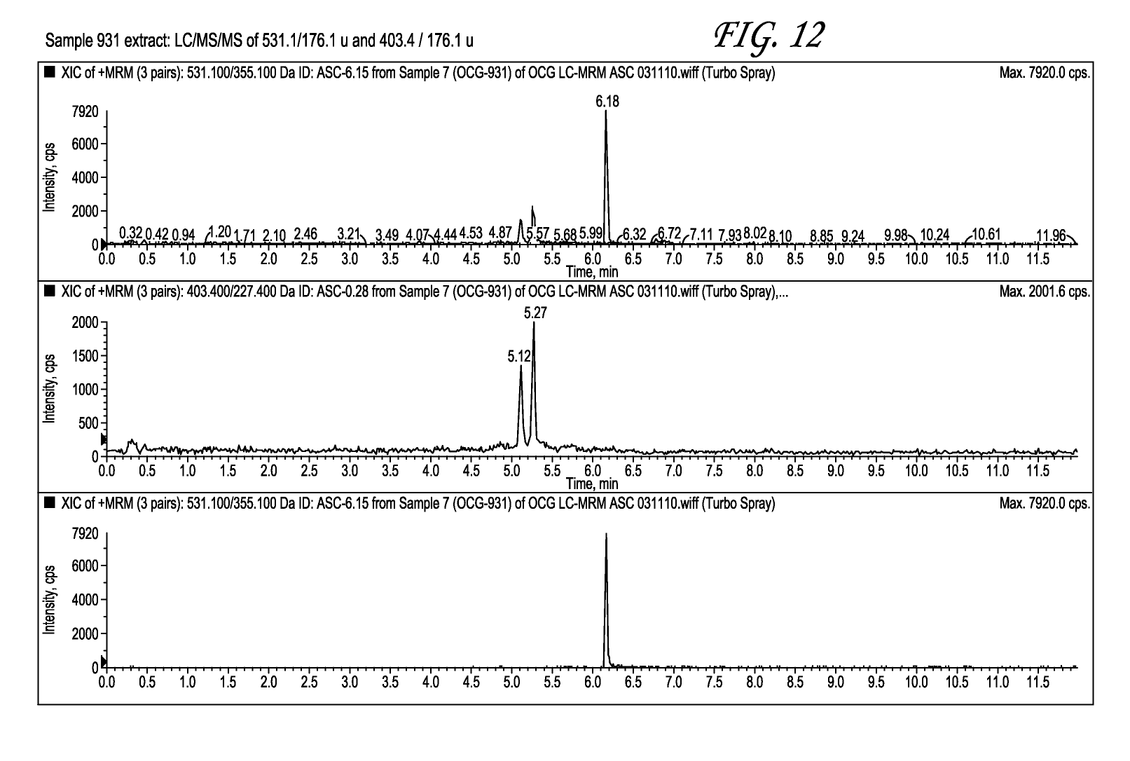

Each sample was assayed under LC/MS/MS to confirm this peak's presence by monitoring the fragmentation (FIG. 11 for sample 930 and FIG. 12 for sample 931) of 403.4 u to 176.1 u and 531 u to 176.1 u.

Both samples contained the peaks at 5.12 and 5.28 minutes of 403.4/174.1 u and at 6.18 minutes of 531.1/176.1 u, thus indicating the presence of ascorbic acid conjugates in which an ascorbic acid is linked to a lipid in eye tissue.

Proposed Mechanism

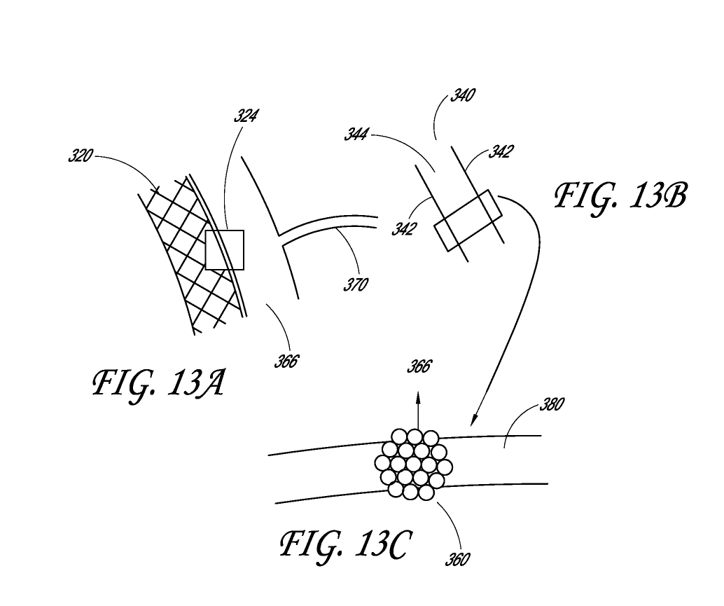

The experiment shows the presence of ascorbate as a component of phospholipid molecules in samples of eye tissue. Without wanting to be bound by any theory, it is believed that phospholipids or other lipids associated with an ascorbic acid or derivative or salt thereof are present in normal eye tissue, specifically in the endothelial layer 324 of Schlemm's canal 366 and/or the trabecular meshwork cells, and may play a role in the maintenance of normal intraocular pressure by regulating drainage of the aqueous humor through the membranes of the JCM cells as part of an osmotic drive. As seen in FIG. 13A, the trabecular meshwork 320 is separated from Schlemm's canal 366 by a single layer of endothelial cells 324. Once the aqueous humor passes through the endothelial layer, it drains into Schlemm's canal and then into the scleral venous system by way of bridging vessels 370. In FIG. 13B, the cell membrane 340 of an endothelial cell is seen with lipophilic regions 342 and hydrophilic regions 344. FIG. 13C shows the endothelial cell membrane 380 with direction of aqueous humor travel indicated by the arrow facing Schlemm's canal 366. A proposed structure in the cell membrane of the endothelial layer 360 is shown as an arrangement of micelles 366, which bridge the cell membrane 340, and serve to transport aqueous humor across the membrane. After traversing the opposing membrane, the aqueous humor is released into Schlemm's canal.

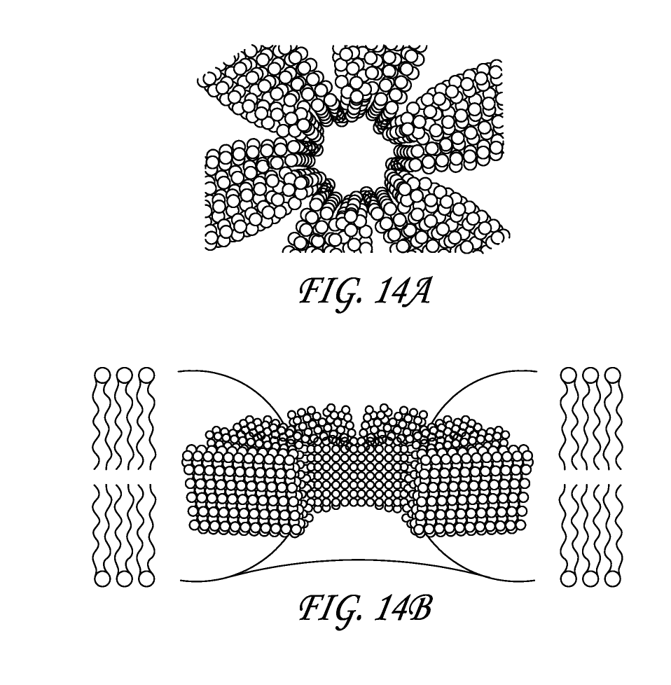

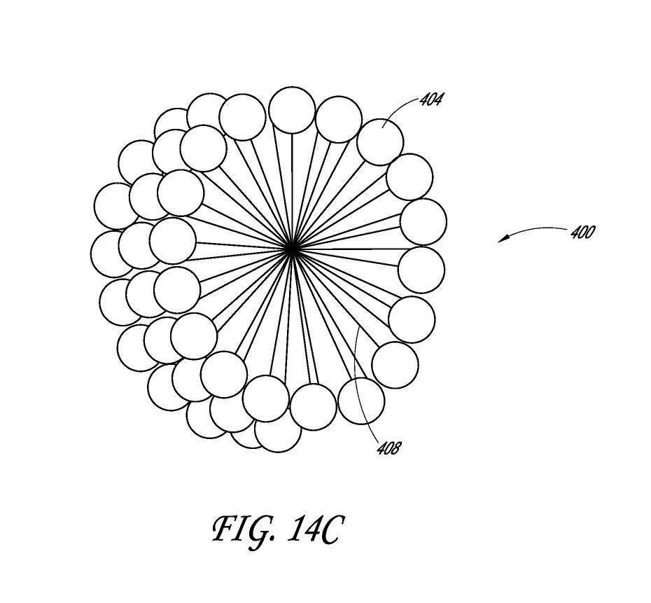

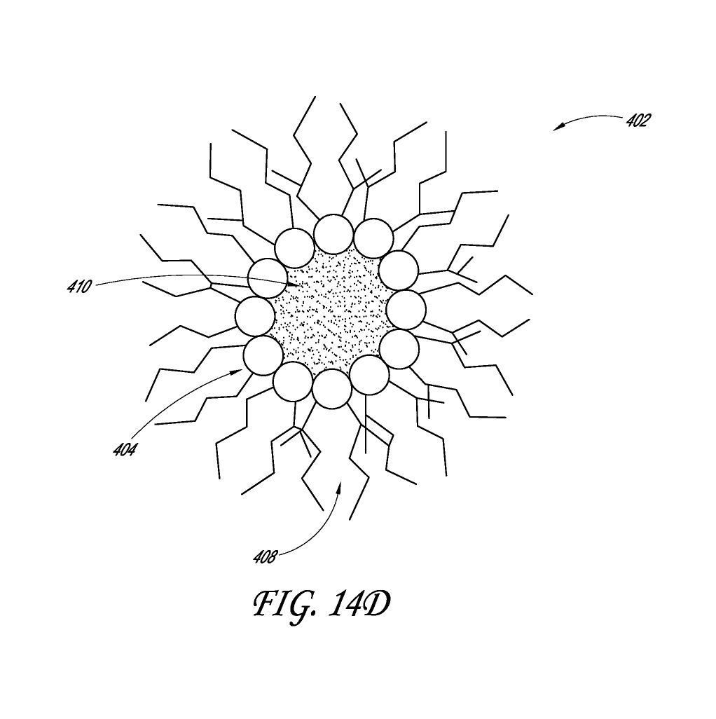

It is proposed that phospholipid molecules or other molecules comprising an ascorbic acid or ascorbic acid derivative head are produced by the specialized cells of the JCM and transported to the cell membranes. The ascorbic acid phospholipids or other molecules may be present in the cell membranes in sufficient concentration such that they may form micelles 400 with the polar (ascorbate) heads 404 facing outward and the hydrophobic tails 408 facing inward (FIG. 14C). These micelles may contain a single type of phospholipid molecule or multiple types of molecules. Localized groupings of these micelles may span the cell membrane as seen in the representative rendering in FIG. 13C. The ascorbic acid heads of adjacent micelles form hydrogen bonds. However, as the volume and pressure of aqueous fluid increase, the inter-micellar bonds may be more easily disrupted and water molecules pass between them, permitting aqueous humor to cross the cell membrane by traveling between spheres. After molecules pass into the cell from the anterior chamber side, the consequent volume and pressure expansion of the cytoplasm causes spheres to form across the cell membrane on the Sclemm's canal side of the cell and the process is repeated, thereby completing the transfer of aqueous humor into the aqueous venous system.

Alternatively or additionally, pinocytotic vesicles that have been observed in the juxtacanalicular meshwork and the inner wall of Schlemm's Canal may represent structures based on ascorbate based phospholids or normally occurring phospholipids. The highly polar environment formed by an increase in the aqueous humor pressure may cause the lipid bilayer of the cell membrane to fold back on itself. In this setting a monolayer or bilayer of the ascorbate based phospholipid may contain aqueous derived fluid solutes and waste products for delivery to Schlemm's canal.

Turning now to FIG. 14D, another possible configuration of the ascorbate based phospholipids or ascorbate based amphipathic species is reverse micelles 402, in which, once sufficient concentrations of the molecules are present in close proximity within the polar environment of the cell membrane, the polar ascorbate heads 404 turn inward, and the fatty acid chains 408 outward, which permits water and solute 410 to be trapped within the core, and thus transported through the cell membrane. This conformation may be difficult to achieve in the normal hydrophobic lipid tail interior of the cell membrane. Without being limited by the disclosed theories, there are many possibilities for this formation, including: (1) Type I or Type II Integral proteins could cluster and attract the hydrophobic tails of these amphipathic molecules into contact with their internal non-polar domains creating a central polar cylindrical core for fluid transport, (2) Type III or IV Integral proteins could also form the same polar channel with a single molecule perhaps more efficiently, and (3) a Beta Barrel configuration could also form a central polar core through the same interactions.

Additionally or alternatively, it is proposed that the ascorbic acid phospholipids produced by the JCM, other ascorbate based amphipathic species, or any other amphipathic species, may form a cylindrical structure, with the polar ascorbate heads internal, bridging the cell membrane as a channel. Ceramides and Sphinosine, which are amphipathic molecules with a lipid tail and polar head, are known to form stable channels within the mitochondrial membrane. (U.S. Pat. No. 7,897,401; Anishkin, A. et al. (2006) “Searching for the Molecular Arrangement of Transmembrane Ceramide Channels,” Biophys. J. 90:2414-2426; Siskind, L. J. et al. (2006) “Ceramide Forms Channels in Mitochondrial Outer Membranes at Physiologically Relevant Concentrations,” Mitochondrion 6(3):118-125 (Epub Mar. 29, 2006); Siskind, L. J. et al. (2005) “Sphingosine Forms Channels in Membranes That Differ Greatly From Those Formed by Ceramide,” J. Bioenerg. Biomembr. 37:227-236, all of which are herein incorporated by reference in their entirety). It is proposed that the ascorbic acid phospholipids may form similar channels, as represented in FIGS. 14A-B. When intraocular pressure is low, the ascorbic acid heads form bonds to each other, and as the intraocular pressure rises, the water molecules compete increasingly effectively at the binding sites, thereby allowing water to pass through the channel. The increasing pressure from surrounding aqueous fluid may also play a mechanical role in distorting the cell membrane, thereby contributing to the dissociation of bonds between polar moieties and the consequent permeability to water molecules. Mechanical forces may also initiate pinocytotic vesicle formation that has been observed in the juxtacanalicular meshwork and the inner wall of Schlemm's Canal. As the intraocular pressure diminishes in response to increased flow, the bonds between polar moieties are increasingly favored over bonds with water molecules, and the flow diminishes, until an equilibrium is reached. The equilibrium may change based on various factors, such as the rate of production of aqueous humor, but will be self-regulating to maintain a desired pressure.

The phospholipid membrane-spanning structures may be a combination of micelles and cylinders, such that a cylindrical channel contains smaller micelles of the same or similar phospholipids. The phospholipid structure may also form tubular arrangement (hexagonal), or any of various cubic phases. More complicated aggregations of phospholipids have also been observed, including rhombohedral, tetragonal and orthorhombic, and ascorbic acid containing phospholipids in these arrangements, alone or in combination, may contribute to the structures involved in water transport across the JCM membrane.

Any of the above-described phospholipid arrays, spanning the cell membrane, may result in a self-regulating osmotic drive for water transport out of the anterior chamber of the eye into Schlemm's canal. When the pressure is balanced, the ascorbic acid moieties will generally bond with each other and water molecules from the aqueous humor will transport between the hydrophilic heads relatively slowly at a steady state rate. However, even small increases or decreases in pressure may cause the establishment of a new equilibrium flow rate.

Open angle glaucoma may result with a failure in the osmotic drive above. As relatively normal concentrations of ascorbic acid have been found to be present in eye tissue of glaucoma patients, absorption, transport, and ingestion of ascorbic acid are not likely causes of failure, and, furthermore would be expected to cause systemic problems related to vitamin C deficiency rather than isolated intraocular pressure elevations. In some patients, failure of normal phosphorylation of ascorbic acid may result from enzyme deficiency, decreased enzyme activity, or other disturbance. In other patients, enzymes which mediate other assembly steps of the phospholipids or other amphipathic molecules which constitute the osmotic drive may have deficiencies or mutations which lead to diminished concentrations of the phospholipids or amphipathic molecules in the cell membranes of JCM cells or lead to phospholipids or amphipathic molecules with decreased ability to form the structures of the osmotic drive. These enzymes may be specific to the cells of the JCM or may exist in other places, in which case the patient may have other manifestations in addition to glaucoma, and therapeutic compounds may treat those manifestations as well. In some patients, other pathologies may also result in the inability of the osmotic drive to assemble within the cell membrane.

### Therapeutics

The compounds disclosed herein may be employed as pharmaceutical agents, provided in therapeutically effective amounts, to effect the treatment of diseases and conditions, particularly open angle glaucoma. The term “treat,” “treating,” or “treatment” as used herein refers to administering a compound or pharmaceutical composition to a subject for prophylactic and/or therapeutic purposes, and includes: (i) preventing a disease from occurring in a subject which may be predisposed to the disease but has not yet been diagnosed as having it; (ii) inhibiting the disease, i.e. arresting its development; or (iii) relieving the disease, i.e. causing regression of the disease.

Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

As used herein, the term “therapeutically effective amount,” “pharmaceutically effective amount” or “effective amount” refers to that amount (at dosages and for periods of time necessary) of a compound which, when administered to a mammal in need thereof, is sufficient to effect treatment (as defined above). The amount that constitutes a “therapeutically effective amount” will vary depending on the compound being administered, the condition or disease and its severity, and the mammal to be treated, its weight, age, etc., but may be determined routinely by one of ordinary skill in the art with regard to contemporary knowledge and to this disclosure.

### Amphipathic Compounds, Phosphorylated Ascorbic Acid, and Other Ascorbic Acid Derivatives

In some embodiments, an individual suffering from glaucoma is treated by administering a therapeutically effective amount of a therapeutic compound which consists of an amphipathic compound, an ascorbic acid derivative having one or more nonpolar groups or chains (e.g., 8-32 carbon fatty acid chains), or an ascorbate linked to a phosphate group. In some embodiments, the phosphate group may be part of a phospholipid, comprising one or more nonpolar groups or chains. In other embodiments, the therapeutic compound consists of an ascorbate derivative linked to a phosphate group, while in still others, the therapeutic compound consists of an ascorbate analog linked to a phosphate group. The ascorbate is not limited with respect to its form, and any known ascorbate or ascorbate derivative can be used. For example, ascorbate, ascorbic acid, or any pharmaceutically acceptable salt, hydrate, and solvate thereof, can be linked to a phosphate group and delivered to a patient in therapeutically effective amounts. Other polar molecules that have a single or multiple sites capable of hydrogen bonding may also be substituted for the ascorbate group. “Solvate” refers to the compound formed by the interaction of a solvent and a compound described herein or salt thereof; suitable solvates are pharmaceutically acceptable solvates including hydrates. The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### Phospholipid

In other embodiments, the therapeutic compound is a phospholipid or phospholipid derivative containing an ascorbic acid head or other amphipathic molecule or compound. For example, in some embodiments, the therapeutic compound may be a phospholipid-type ascorbic acid derivative resulting from binding a glycerol ester or ether to ascorbic acid via a phosphoric acid residue. Ascorbate can be linked at its 6 position to a phospholipid described herein or to a hydrophilic polymer-lipid conjugate described herein using methods known in the art. For example, the ascorbate can be linked to a phospholipid via a covalent bond, such as by a sulfur atom, an oxygen atom, a nitrogen atom, or a hydrocarbon linking group, using known techniques. In particular instances, about 10% to about 100% of the phospholipids of the micelle or other water transport structure are attached to ascorbate. For example, about 20% to about 95%, about 30% to about 90%, about 40% to about 80%, about 50% to about 95%, about 60% to about 90%, about 70% to about 100%, or about 80% to about 95% of the phospholipids are attached to ascorbate. Method of synthesis may include those disclosed in U.S. Pat. No. 5,098,898, herein incorporated by reference in its entirety.

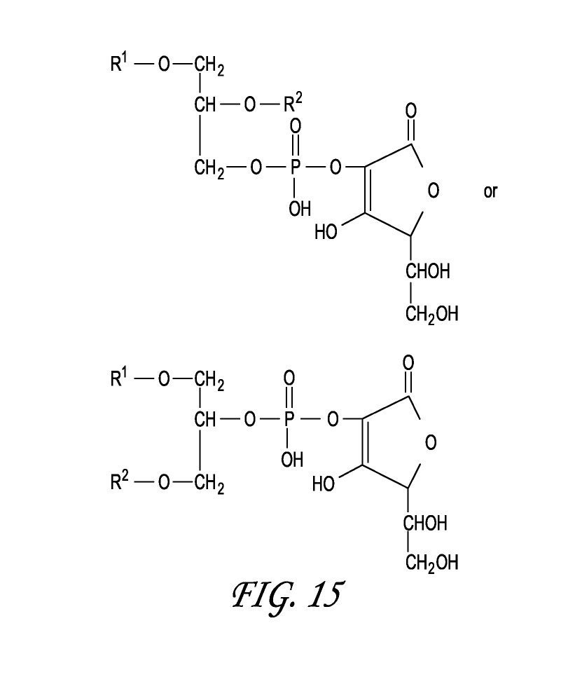

In one aspect thereof, the therapeutic compound may be one of the phospholipid derivatives of the formula shown in FIG. 15, wherein R1 and R2 may be the same or different and each represents an alkyl or acyl group. It is to be noted that neither formula represent any specific configuration nor conformation.

In formulas [I] and [II], the alkyl or acyl group represented by R1 and/or R2 preferably contains 1 to 18 carbon atoms. The carbon chain in the alkyl group may be straight, branched, or cyclic and may contain a cyclic portion. As non-limiting examples of the alkyl group, there may be mentioned lower alkyl groups, such as methyl, ethyl, n-propyl, i-propyl, n-butyl, t-butyl, sec-butyl, n-pentyl, 1-ethylpropyl and i-pentyl, as well as higher alkyl groups, such as n-decyl, n-undecyl, n-pentadecyl, n-hexadecyl, n-heptadecyl, n-octadecyl, and isomeric forms of these. The acyl group when it is an aliphatic acyl may be straight, branched, or cyclic and may contain a cyclic portion. Non-liming examples of acyl groups include acyclic acyl groups, such as acetyl and propionyl, and cyclic acyl groups, such as cyclopentylcarbonyl and cyclohexylcarbonyl. The acyl group may also be an aromatic or araliphatic acyl group, such as benzoyl or phenylacetyl.

The resulting phospholipid may be, for example, 1,2-O-Distearoyl-3-glycerophopsphoryl-ascorbic acid; 1,2-O-Dipalmitoyl-3-glycerophosphorylascorbic acid; 1,2-O-Dihexadecyl-3-glycerophosphorylascorbic acid, 1,2-O-Dilauroyl-3-glycerophosphorylascorbic acid potassium salt; 1-3-O-Dilauroyl-2-glycerolphophorylascorbic acid potassium salt; or 1,3-O-Diethyl-2-glycerophosphorylascorbic acid. These examples are not meant to be limiting, and the invention is intended to encompass other ascorbic acid-phospholipids exhibiting the desired characteristics and behavior in ocular tissue.

The length and composition of the fatty acid tails may vary. For example, the total length may be 18, 20, 22, 24, 26, 28, or 30 carbons, with each tail having 9, 10, 11, 12, 13, 14, or 15 carbons. In other embodiments, each tail may have 7 or 8 carbons. In some embodiments, each tail may have 16, 17, 18, or more carbons. In some embodiments, the fatty acid tails may be saturated or unsaturated. The two tails may be of similar length and/or composition or different length and/or composition.

The phospholipid compound may comprise molecules from a single species of phospholipid, or comprise a mixture of two or more different species of phospholipids. The phospholipids may include those derived from either glycerol (such as phosphoglycerides and glycerophospholipids) or sphingosine (sphingolipids). In certain embodiments, the phospholipids may be triglyceride derivatives in which one fatty acid has been replaced by a phosphorylated group and one of several nitrogen-containing molecules. The fatty acid chains are hydrophobic (as in all fats). However, the charges on the phosphorylated and amino groups make that portion of the molecule hydrophilic, resulting in an amphipathic molecule.

### Micelle

In still other embodiments, the therapeutic compound comprises a micelle 400 or other three dimensional structure, as disclosed above, synthesized with phospholipids containing ascorbate or ascorbate-equivalent sidechains or combinations thereof such that the hydrophobic tails 408 are sequestered in the core and the hydrophilic heads 404 extend away from the center. These may be delivered in high concentrations in aqueous solution using known methods. In some embodiments, the amphipathic molecules comprising an ascorbate (or derivative or equivalent) may be delivered as individual molecules in solution (not already formed into the three dimensional aggregates), wherein the amphipathic molecules are configured to form micelles after delivery, under favorable conditions. For example, micelle formation in situ is typically favored under conditions such as when the concentration of a surfactant is greater than the critical micelle concentration. In other embodiments, micelles or other three dimensional aggregations consisting of other amphipathic molecules which may be taken up and incorporated into the cell membrane of cells of the JCM are provided in therapeutically effective amounts. The micelle or three dimensional aggregations may comprise one or more amphipathic molecules. In some embodiments, the micelles or other three dimensional aggregations of one or more amphipathic molecules may be provided in a stabilized state by use of various stabilizers which would be known to one of skill in the art. The hydrophilic heads of the micelles or other three dimensional aggregations may incorporate a targeting unit capable of selectively binding to a specific cell type and/or tissue. The targeting unit may be a moiety having the capacity to selectively associate with the specific target cell and/or tissue. Thus, the targeting unit may facilitate specific delivery of the micelle of the invention to the target JCM cells and/or eye tissue while minimizing any possible side effects resulting from delivery to non-target cells and/or tissues. Targeting units include, but are not restricted to antibodies, ligands, substrates, nucleic acid molecules such as RNA, DNA, PNA or other molecules that bind specifically to a cell and/or tissue. The targeting unit may be covalently attached directly via a covalent bond formed between functional groups present on the targeting unit and the external surface of the micelle, or, alternatively, attachment may involve a linker.

### Enzyme

In other embodiments, an enzyme having an enzymatic activity of forming an amphipathic compound is administered in therapeutically effective amounts for uptake into the eye. In some embodiments, the enzyme may be a kinase that is capable of phosphorylating ascorbate. For example, phospholipase D can be used to synthesize 6-Phosphatidyl-L-ascorbic acid as described by Nagao et al. in Lipids 26:390-94 (1991), herein incorporated by reference in its entirety. Phospholipase D from Streptomyces lydicus may be obtained or the enzyme may be synthesized in a lab, both of which can be accomplished via methods known in the art. Other enzymes which are effective for phosphorylating ascorbate may be synthesized or isolated and administered to a subject in therapeutically effective amounts. In some embodiments, the enzyme may be an esterase. As a non-limiting example, where an amphipathic molecule has been modified by the addition of a cleavable ester group (e.g. to increase transport into the eye), the esterase may cleave an ester linkage to release the amphipathic molecule.

### Gene Therapy

In still other embodiments, treatment consists of gene therapy, in which one or more of the therapeutic agents is a nucleic acid that encodes a therapeutic agent. For example, the nucleic acid may encode for a protein or peptide. The protein or peptide may comprise afunctional kinase enzyme to phosphorylate ascorbic acid, or to phosphorylate an ascorbate derivative or otherwise contribute to the production of ascorbic acid, ascorbate derivate or ascorbate equivalent phospholipids in operable association with regulatory elements sufficient to direct expression of the nucleic acid is administered to the eye. As another non-limiting example, the nucleic acid may encode a protein or peptide having esterase activity, wherein the functional esterase enzyme may cleave ester groups and release amphipathic molecules. A composition comprising a nucleic acid therapeutic can consist essentially of the nucleic acid or a gene therapy vector in an acceptable diluent, or can comprise a drug release regulating component such as a polymer matrix with which the nucleic acid or gene therapy vector is physically associated; e.g., with which it is mixed or within which it is encapsulated or embedded. The gene therapy vector can be a plasmid, virus, or other vector. Alternatively, the pharmaceutical composition can comprise one or more cells which produce a therapeutic nucleic acid or polypeptide. Preferably such cells secrete the therapeutic agent into the extracellular space.

Viral vectors that have been used for gene therapy protocols include, but are not limited to, retroviruses, lentiviruses, other RNA viruses such as poliovirus or Sindbis virus, adenovirus, adeno-associated virus, herpes viruses, SV 40, vaccinia and other DNA viruses. Replication-defective murine retroviral or lentiviral vectors are widely utilized gene transfer vectors. Chemical methods of gene therapy involve carrier-mediated gene transfer through the use of fusogenic lipid vesicles such as liposomes or other vesicles for membrane fusion. A carrier harboring a nucleic acid of interest can be conveniently introduced into the eye or into body fluids or the bloodstream. The carrier can be site specifically directed to the target organ or tissue in the body. Cell or tissue specific DNA-carrying liposomes, for example, can be used and the foreign nucleic acid carried by the liposome absorbed by those specific cells. Gene transfer may also involve the use of lipid-based compounds which are not liposomes. For example, lipofectins and cytofectins are lipid-based compounds containing positive ions that bind to negatively charged nucleic acids and form a complex that can ferry the nucleic acid across a cell membrane.

Delivery of gene therapy may also be accomplished via cationic polymers. Certain cationic polymers spontaneously bind to and condense nucleic acids such as DNA into nanoparticles. For example, naturally occurring proteins, peptides, or derivatives thereof have been used. Synthetic cationic polymers such as polyethylenimine (PEI), polylysine (PLL) etc. condense DNA and are useful delivery vehicles. Dendrimers can also be used. Many useful polymers contain both chargeable amino groups, to allow for ionic interaction with the negatively charged DNA phosphate, and a degradable region, such as a hydrolyzable ester linkage. Examples include poly(alpha-(4-aminobutyl)-L-glycolic acid), network poly(amino ester), and poly(beta-amino esters). These complexation agents can protect nucleic acids against degradation, e.g., by nucleases, serum components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid.

### Other

In still other embodiments, treatment consists of synthesizing an artificial membrane to replace some or all of the endothelial lining of Schlemm's canal, in some embodiments of which synthesized micelles or other phospholipid membrane spanning arrangements are interspersed. This membrane may be surgically implanted after excision of some or all of the endothelial lining.

### Individual Treatment

In some embodiments, the treatment of glaucoma is tailored to the individual patient. Multiple variations of the compound with different polar moieties, phosphate groups, glycerol equivalents, fatty acid chains, or diglyceride groups are provided, wherein administration of each variation results in characteristic reduction of intraocular pressure or a characteristic pressure at equilibrium. Methods of treatment may include the measurement of intraocular pressure prior to administration of the therapeutic agent, selection of compound based on the desired reduction in intraocular pressure or target pressure, and administration of that compound. The intraocular pressure may be monitored during therapy and different agents or a combination of different agents may be selected to maintain a desired pressure; for example, between 10 and 20 mm Hg, or sometimes between about 15 to about 18 mm Hg. After the selection of such different agents or combination of different agents, they may then be administered.

### Methods of Administration

Amphipathic molecules and compounds or their precursors that increase transport of aqueous humor may be modified in an effort to increase the ability of the molecule to enter the eye. Examples may include, but are not limited to, the addition of cleavable ester groups or other easy leaving groups and molecules/compounds alterable by native enzymes or metabolic pathways into the intended amphipathic molecules capable of increasing transport of aqueous humor.

Various methods of administering the therapeutic compounds systematically are contemplated. These include topical administration to the eye via drops, spray, gel, ointment, or other vehicle. The active compounds disclosed herein are administered to the eyes of a patient by any suitable means, but preferably administered by administering a liquid or gel suspension of the active compound in the form of drops, spray or gel. Alternatively, the active compounds are applied to the eye via liposomes. Further, the active compounds can be infused into the tear film via a pump-catheter system. Another embodiment involves the therapeutic compound contained within a continuous or selective-release device, for example, membranes such as, but not limited to, those employed in the Ocusert™ System (Alza Corp., Palo Alto, Calif.). As an additional embodiment, the active compounds can be contained within, carried by, or attached to contact lenses, which are placed on the eye. Another embodiment of the invention involves the therapeutic compound contained within a swab or sponge, which is applied to the ocular surface. Another embodiment of the invention involves the therapeutic compound contained within a liquid spray, which is applied to the ocular surface.

In other embodiments, the therapeutic compound is delivered by intraocular injection performed periodically. In some embodiments, the therapeutic compounds may be administered via subconjunctival injection, in others through intracameral (anterior chamber), intravitreal or subscleral injection. The therapeutic compound may be delivered directly to Schlemm's canal via catheter or implanted shunt. Further means of systemic administration of the active compound would involve direct intra-operative instillation of a gel, cream, or liquid suspension form of a therapeutically effective amount of the therapeutic compound. In some embodiments, the therapeutic compounds are administered in a suspension. In some embodiments, the therapeutic compounds may be administered, for example, by sustained release implants and microspheres for intracameral or anterior vitreal placement within a biodegradable polymer that releases a therapeutic amount of the compound over a period of time ranging up to a year or more. Additionally, in some embodiments, the therapeutic compounds may be administered by an implanted drug delivery system which releases a therapeutically effective amount of the compound over time. Implantation of the drug delivery system may be surgical or via injection.

The topical solution containing the therapeutic compound can also contain a physiologically compatible vehicle, as those skilled in the ophthalmic art can select using conventional criteria. The vehicles can be selected from the known ophthalmic vehicles which include, but are not limited to, saline solution, water polyethers (such as polyethylene glycol), polyvinyls (such as polyvinyl alcohol and povidone), cellulose derivatives (such as methylcellulose and hydroxypropyl methylcellulose), petroleum derivatives (such as mineral oil and white petrolatum), animal fats (such as lanolin), polymers of acrylic acid (such as carboxypolymethylene gel), vegetable fats (such as peanut oil) and polysaccharides (such as dextrans), and glycosaminoglycans (such as sodium hyaluronate), and salts (such as sodium chloride and potassium chloride).

In addition to the topical method of administration described above, there are various methods of administering the therapeutic compounds of the invention systemically. One systemic method of administration may involve an aerosol suspension of respirable particles comprised of the active compound, which the subject inhales. The therapeutic compound is absorbed into the bloodstream via the lungs and subsequently contact the ocular tissues in a pharmaceutically effective amount. The respirable particles are a liquid or solid, with a particle size sufficiently small to pass through the mouth and larynx upon inhalation; in general, particles ranging from about 1 to 10 microns, but more preferably 1-5 microns, in size are considered respirable.

Another means of systemically administering the active compounds to the eyes of the subject would involve administering a liquid/liquid suspension in the form of eye drops or eye wash or nasal drops of a liquid formulation, or a nasal spray of respirable particles which the subject inhales. Liquid pharmaceutical compositions of the active compound for producing a nasal spray or nasal or eye drops can be prepared by combining the active compound with a suitable vehicle, such as sterile pyrogen free water or sterile saline by techniques known to those skilled in the art.

Other means of systemic administration of the therapeutic compound may involve oral administration, in which pharmaceutical compositions containing active compounds are in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use are be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Additional means of systemic administration of the therapeutic compound to the eyes of the subject would involve a suppository form of the active compound, such that a therapeutically effective amount of the compound reaches the eyes via systemic absorption and circulation.

### Drug Screening Method

A method for screening compounds for use in glaucoma treatment relates to identifying compounds which are taken up by the JCM and are incorporated into the cell membrane. One aspect of the invention relates to methods of identifying novel compounds capable of affecting water transport in the eye.

Fresh donor eye tissue from normal eyes or eyes with glaucoma may be used to create a framework for screening compounds. In some embodiments, the drug screening method may include the following. The anterior segment of the eye with a scleral rim of about 3-4 mm may be resected. The uveal tissue may be removed from the internal surface of the eye. The remaining anterior eye tissue may be clamped to a holding device. Fluid may be perfused into the bare anterior chamber. This process removes other means of outflow from the anterior chamber; therefore, the only remaining outflow tract is through the trabecular meshwork, Schlemm's canal, and the intra-scleral aqueous collector channels. A candidate therapeutic compound may then be infused into the anterior chamber, allowed to incorporate into the cell membranes, and various measurements performed. Such measurements include the measurement of the amount or rate of uptake of the candidate therpauetic molecule into the donor eye tissue. For example, pressure decay curves or flow rates may be measured to determine whether a candidate therapeutic compound has a favorable effect on facility of outflow.

This model may also be used to measure the effectiveness of the candidate therapeutic compound by studying changes in facility of outflow before and after the compound is introduced. The Goldman equation may be used, for example, to measure facility of outflow, which should increase as the function of the trabecular meshwork increases:

Goldmann equation:Po=(F/C)+Pv

Where Po=observed pressure; F=formation rate of aqueous; C=facility of outflow; Pv=episcleral venous pressure.

In the drug testing set up, Pv is zero as there are no aqueous veins and C is in reality made up of trabecular meshwork resistance and additional contributions such as resistance within the intrascleral aqueous humor collector channels. These additional factors remain constant for a particular eye. Thus changes in trabecular function in the living, perfused trabecular tissue may be deduced based on changes in observed pressure/flow responses.

For example, an ex vivo anterior segment perfusion culture device which functions as detailed above was first described by Douglas (Johnson, D., Invest Ophthalmol Vis Sci 1987; 28:945-953, herein incorporated by reference in its entirety. The model has also been used to evaluate changes in outflow with the addition of drugs such as dexamethasone (Clark A F, “Dexamethasone-Induced ocular hypertension in perfusion cultured human eyes,” Invest Ophthalmol Vis Sci; 36:478-489 (1995), herein incorporated by reference in its entirety) and the efficacy of surgical treatment for the trabecular meshwork; i.e., stents. However, a novel use is proposed which may include, in some embodiments:

(a) introducing a candidate therapeutic compound, such as a candidate lipid based amphipathic molecule or other amphipathic molecule/compound or ascorbate phosphate group conjugate into the anterior segment of the donor tissue, and
(b) determining the uptake of the candidate molecule/compound into the eye tissue.

In some embodiments, the use may comprise:

(a) introducing a candidate therapeutic compound, such as a candidate lipid based amphipathic molecule/compound or ascorbate-phosphate group conjugate into the anterior segment of the donor tissue; and

(b) determining whether the presence of said candidate therapeutic compound, such as a candidate lipid based amphipathic molecule/compound or ascorbate-phosphate group conjugate, affects the rate of outflow from the anterior segment of the eye or the pressure maintained in the anterior segment.

These embodiments are intended to be illustrative of the drug screening methods contemplated, and are not intended to limit the scope of the invention. Additional steps may be added or the steps disclosed above taken in a different order without departing from the invention.

Although embodiments and methods have been disclosed in the context of glaucoma treatment, it will be understood by those skilled in the art that embodiments and methods disclosed herein may also be used in other contexts. For example, administration of ascorbic acid linked to phosphate group, phospholipid chains, synthesized micelles, and/or membrane implant may be utilized in therapeutic amounts to treat other disorders in which fluid outflow regulation is dysfunctional. Examples in which fluid outflow regulation utilizing disclosed therapeutic methods include, but are not limited to, the treatment of hydrocephalus and in an artificial kidney.

Although this has been disclosed in the context of certain preferred embodiments and examples, it will be understood by those skilled in the art that the invention extends beyond the specifically disclosed embodiments to other alternative embodiments and/or uses of the invention and obvious modifications and equivalents thereof. In addition, while the number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments can be made and still fall within the scope of the invention. Accordingly, it should be understood that various features and aspects of the disclosed embodiments can be combined with, or substituted for, one another in order to perform varying modes of the disclosed invention. Thus, it is intended that the scope of the invention herein disclosed should not be limited by the particular disclosed embodiments described above, but should be determined only by a fair reading of the claims.

What is claimed is:

1. A method for screening candidate molecules for therapeutic effect, the method comprising:
perfusing an anterior chamber resected from donor eye tissue with a perfusion fluid, such that outflow of the perfusion fluid from the anterior chamber occurs through the trabecular meshwork, Schlemm's canal, and the intra-scleral aqueous collector channels;
introducing a candidate molecule into the anterior chamber, wherein the candidate molecule is selected from ascorbic acid conjugates, phosphorylated ascorbic acid, phosphorylated ascorbic acid derivatives, ascorbic acid containing phospholipids, ascorbic acid derivatives containing phospholipid, ascorbic acid containing glycolipid, ascorbic acid derivatives containing glycolipid, or other amphipathic molecules; and
determining one or more measurements of therapeutic effect selected from an amount of candidate molecule uptake into donor eye tissue, a rate of candidate molecule uptake into donor eye tissue, a pressure maintained in the anterior chamber, or a rate of outflow of perfusion fluid from the anterior chamber.

2. The method of claim 1, further comprising:
determining baseline values for the one or more measurements of therapeutic effect after perfusing the anterior chamber with the perfusion fluid, and before introducing the candidate molecule;
determining a second set of values for the one or more measurements of therapeutic effect after introducing the candidate molecule; and
determining whether introducing the candidate molecule resulted in a change from the baseline values.

3. The method of claim 2, wherein the one or more measurements is the rate of outflow of perfusion fluid from the anterior chamber.

4. The method of claim 2, wherein the one or more measurements is the pressure maintained in the anterior chamber.

5. The method of claim 1, further comprising determining the one or more measurements of therapeutic effect using anterior chambers resected from normal eyes and eyes with glaucoma.
